(19) United States
(12) Patent Application Publication
(10) Pub. No.: US 2018/0092859 A1
(43) Pub. Date: Apr. 5, 2018

(54) A Supramolecular Chitosan Complex Drug Delivery Platform

(71) Applicant: The Research Foundation for the State University of New York, Albany, NY (US)

(72) Inventors: Ivan Gitsov Ivanov, Syracuse, NY (US); Yizhi Meng, Centereach, NY (US)

(21) Appl. No.: 15/562,526

(22) PCT Filed: Mar. 31, 2016
(86) PCT No.: PCT/US16/25250

§ 371 Date: Sep. 28, 2017

Related U.S. Application Data
(60) Provisional application No. 62/142,000, filed on Apr. 2, 2015.

(51) Int. Cl. A61K 9/51; C07K 17/02; A61K 31/704; B82Y 5/00; A61K 31/337; A61K 31/5365; A61K 39/395; A61K 47/55; A61K 47/61

(52) U.S. Cl. CPC A61K 9/5161; C07K 17/02; A61K 31/704; B82Y 5/00; A61K 31/337; A61K 2039/625; A61K 39/39558; A61K 47/55; A61K 47/61; C12Q 2600/158; C12Q 2600/16; A61K 31/5365

(57) ABSTRACT

The subject of this invention is a supramolecular (micellar system) with hydrophilic shell of linear aliphatic poly(ether) and hydrophobic core of perfectly branched poly(benzyl ether). The system can bind a broad variety of hydrophobic substances ranging from small spherical molecules to modestly large linear chains without the necessity of chemical bond formation. It is non-toxic and fully biocompatible.

### FIELD OF THE INVENTION

The present invention is related to compositions and methods of use for a supramolecular chitosan complex system (e.g., for example, a micellar system) with a hydrophilic shell of a linear aliphatic poly(ether) polymer and a chitosan hydrophobic core. Further, the chitosan hydrophobic core comprises a plurality of branched poly(benzyl ether) polymers. In some systems, the poly(benzyl ether) polymers are perfectly branched. The chitosan hydrophobic core of the supramolecular complex system can bind hydrophobic substances ranging including, but not limited to, small spherical molecules and/or modestly large linear chains.

### BACKGROUND

Copolymers that form well defined micelles in aqueous media have been known since 1991 and can encapsulate water-insoluble substances in their hydrophobic interior. Further studies with polyaromatic hydrocarbons demonstrated encapsulation of substances such as fluorescent biomarkers, drugs and/or oligopeptides in to the hydrophobic interior of such micelles.

What is needed in the art is a copolymer micelle having a multi-functional nano-structure to provide a platform for early diagnosis and drug delivery.

### SUMMARY OF THE INVENTION

The present invention is related to compositions and methods of use for a supramolecular chitosan complex system (e.g., for example, a micellar system) with a hydrophilic shell of a linear aliphatic poly(ether) polymer and a chitosan hydrophobic core. Further, the chitosan hydrophobic core comprises a plurality of branched poly(benzyl ether) polymers. In some systems, the poly(benzyl ether) polymers are perfectly branched. The chitosan hydrophobic core of the supramolecular complex system can bind hydrophobic substances ranging including, but not limited to, small spherical molecules and/or modestly large linear chains.

In one embodiment, the present invention contemplates a supramolecular complex comprising a hydrophobic core encapsulated by a hydrophilic polymer shell, wherein said hydrophobic core comprises at least one chitosan nanoparticle anchored by at least one linear-dendritic co-polymer. In one embodiment, the chitosan is a covalently modified chitosan. In one embodiment, the hydrophobic core further comprises an unattached compound. In one embodiment, the unattached compound includes, but is not limited to, a drug, a cell marker or a biosensor. In one embodiment, the hydrophilic polymer shell comprises a targeting agent. In one embodiment, the targeting agent comprises avidin and a streptavidin-conjugated antibody. In one embodiment, the streptavidin-conjugated antibody has specific affinity for a disease biomarker. In one embodiment, the disease biomarker includes, but is not limited to, a protein, a peptide, a polypeptide or nucleic acid sequence.

In one embodiment, the present invention contemplates a method, comprising: a) providing: i) a supramolecular complex comprising a hydrophobic core encapsulated by a hydrophilic polymer shell, wherein said hydrophobic core comprises at least one chitosan nanoparticle anchored by at least one linear-dendritic co-polymer and a compound; ii) a patient exhibiting at least one symptom of a medical condition; and b) administering said supramolecular complex to said patient under conditions such that said at least one symptom is reduced. In one embodiment, the medical condition comprises a metastatic cancer. In one embodiment, the metastatic cancer includes, but is not limited to, an osteosarcoma or a breast cancer. In one embodiment, the hydrophilic shell further comprises a targeting agent specific for a biomarker of said medical condition. In one embodiment, the targeting agent comprises an antibody having specific affinity for said biomarker. In one embodiment, the compound includes, but is not limited to, a drug, a cellular marker or a biosensor. In one embodiment, the drug includes, but is not limited to, doxorubicin, ansamitocin-P3 or paclitaxel. In one embodiment, the administering comprises an intracellular delivery.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement.

The term “supramolecular complex” as used herein, refers to any combination of different molecules of interest wherein the combination has a high molecular weight. For example, some complexes having a high molecular weight would be comprised of a combination of polymers greater than 100 units long and a chitosan nanoparticle.

The term “chitosan” as used herein, refers a linear polysaccharide polymer comprising units of randomly distributed β-(1-4)-linked D-glucosamine (e.g., a deacetylated unit) and N-acetyl-D-glucosamine (e.g., an acetylated unit).

The term “hydrophobic core” as used herein, refers to a non-aqueous central region of a supramolecular complex comprises of hydrophobic polymers and chitosan nanoparticles that repel aqueous soluble compounds and attract non-aqueous soluble compounds. Preferably, the hydrophobic core is a spherical fullerene molecule.

The term “fullerene molecule” as used herein, is a molecule of carbon in the form of a hollow sphere, ellipsoid, tube, and many other shapes. Spherical fullerenes are also called buckyballs, and they resemble soccer balls (e.g., for example, Fullerene-C₆₀, Sigma-Aldrich). Cylindrical fullerenes also may be called carbon nanotubes or buckytubes. Fullerenes are similar in structure to graphite, which is composed of stacked graphene sheets of linked hexagonal rings; but they may also contain pentagonal (or sometimes heptagonal) rings.

The term “hydrophilic shell” as used herein, refers to a molecular layer of hydrophilic polymers that attract aqueous soluble compounds and repel non-aqueous soluble compounds. Usually, the hydrophilic shell encapsulates and stabilizes a hydrophobic chitosan core to form a supramolecular complex.

The term “linear-dendritic co-polymer” as used herein, refers to an amphiphilic polymer comprising a hydrophobic region (e.g., containing poly(ethylene glycol) or poly(ethylene oxide) of molecular weight 3,000 to 15,000 Da and dendritic poly(3,5-dihydroxybenzyl alcohol) of molecular weight 728 and 1577 Da.) and a hydrophilic region (e.g., a poly(ethylene oxide) chain). Some linear-dendritic co-polymers may be perfectly branched while other linear-dendritic co-polymers may be imperfectly branched.

The term “perfectly branched” as used herein, refers to any branched macromolecule (e.g., for example, a dendritic macromolecule) which does not have any structural defects, including, but not limited to, missing polymer branches in the entire structure of the macromolecule from the center to the periphery.

The term “imperfectly branched” as used herein, refers to any branched macromolecule (e.g., for example, a dendritic macromolecule) which does have at least one structure defect including, but not limited to, a missing polymer branch in the entire structure of the macromolecule from the center to the periphery.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc.) to a binding partner under conditions such that the binding partner becomes unresponsive to its natural ligands. Inhibitory compounds may include, but are not limited to, small organic molecules, antibodies, and proteins/peptides.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “unattached” as used herein, refers to any absence of interaction between a medium (or carrier) and a drug. Such unattachment includes the absence of, but is not limited to, covalent bonding, ionic bonding, Van der Waals forces or friction, and the like. A drug is unattached to a medium (or carrier) if it is freely solubilized within the carrier or medium.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “delivered”, “delivering”, “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long term inflammatory effects such that the molecular structure has not been altered. In one sense, bioerosion represents the final stages of “biodegradation” wherein stable low molecular weight products undergo a final dissolution.

The term “bioresorbable” as used herein, refers to any material that is assimilated into or across bodily tissues. The bioresorption process may utilize both biodegradation and/or bioerosion.

The term “biostable” as used herein, refers to any material that remains within a physiological environment for an intended duration resulting in a medically beneficial effect.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “luminescence” and/or “fluorescence”, as used herein, refers to any process of emitting electromagnetic radiation (light) from an object, chemical and/or compound. Luminescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including chemical, thermal, electrical, magnetic, electromagnetic, physical, or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents one embodiment of a supramolecular chitosan complex loaded with a plurality of biologically active substances. Black Sphere: Hydrophobic chitosan nanoparticle. Yellow circles: A biologically active substance. Green oval: A cellular marker. Blue-Red polymer: Amphiphilic Linear-dendritic copolymers comprising hydrophobic dendrons (red) and hydrophilic poly(ethylene oxide) chains (red). Tethered orange circle/blue spheres: A first targeting agent (e.g., avidin/biotinylated antibody complex). Overlapping flat orange discs: 5-β-cholanic acid.

FIG. 2 presents exemplary data showing the viability of MG63 human osteosarcoma cells exposed for 3 days to hydrophobically modified glycol chitosan (HGC).

FIG. 3 present exemplary data showing confocal microscopy images of MG63 human osteosarcoma cells (nuclei visualized in blue channel) after delivery of hydrophobically modified chitosan nanomicelles (red channel) FIG. 4 presents exemplary data showing confocal microscopy images of astrocyte 1321N1 cell culture labeling using a supramolecular complex encapsulating an acrylodan-labeled C-terminal phospholipase D (PLD) peptide. Both Control and Labeled Astrocyte images were taken at 400× magnification using conventional light microscopy with a DAPI filter set.

FIG. 4A: A control astrocyte cell population image that was exposed twice to achieve even faint picture of cells. Added glycerol caused a faint auto-fluorescence as seen in these control cells.

FIG. 4B: A labeled astrocyte cell population image subsequent to the intracellular delivery of a supramolecular chitosan complex encapsulating acrylodan-labeled C-terminal PLD peptide. The image demonstrates highly distinct images of the astrocyte cell culture cell population.

FIG. 5 presents exemplary data showing the viability of 4T1 murine carcinoma cells exposed to doxorubicin encapsulated in hydrophobically modified glycol chitosan (HGC) nanoparticles or HGC-linear dendritic copolymer nanocomplexes.

### DETAILED DESCRIPTION OF THE INVENTION

The present invention is related to compositions and methods of use for a supramolecular chitosan complex system (e.g., for example, a micellar system) with a hydrophilic shell of a linear aliphatic poly(ether) polymer and a chitosan hydrophobic core. Further, the chitosan hydrophobic core comprises a plurality of branched poly(benzyl ether) polymers. In some systems, the poly(benzyl ether) polymers are perfectly branched. The chitosan hydrophobic core of the supramolecular complex system can bind hydrophobic substances ranging including, but not limited to, small spherical molecules and/or modestly large linear chains.

In one embodiment, the present invention contemplates a supramolecular complex system comprising a plurality of linear-dendritic copolymers and a plurality of chitosan nanoparticles. In one embodiment, the present invention contemplates a method comprising a drug delivery supramolecular complex system for theranostic applications.

At the core of some embodiments of the presently disclosed technology is a supramolecular (micellar) system comprising a hydrophilic shell of linear aliphatic poly(ether) and hydrophobic core of branched (dendritic) poly(benzyl ether) (e.g., for example, perfectly branched). The system can bind a broad variety of hydrophobic substances ranging from small spherical molecules (e.g., for example, Fullerene-C₆₀, Sigma-Aldrich) to relatively large macromolecules such as poly(saccharides) without the necessity of chemical (covalent) bond formation. It is non-toxic and fully biocompatible.

### I. Drug Delivery Systems

The success of drug delivery systems largely depends on their ability to accumulate in the target tissues with minimal loss while in the blood circulation, and the controlled release of the therapeutic inside the cells [1, 2]. For example, reducing the size of the vehicles from micro- to nano-sized (100-200 nm) can slow down the rapid clearance from the circulation by macrophages of the reticuloendothelial system (RES) following intravenous administration [3]. To achieve targeted delivery, various delivery systems have utilized the targeting capabilities of nanocarriers [4, 5]. This passive strategy exploits the enhanced permeability and retention (EPR) of the diseased tissue microenvironment due to its rapid growth that prevents the formation of fully functional vasculature and proper lymphatic drainage [1, 2, 6]. To promote site-specific targeted release of therapeutics, one strategy is to design stimuli responsive micelles that are very sensitive to intracellular pH [7] and enzymatic activity [8]. Often this increases the therapeutic efficacy of the payload, thus reducing the therapeutic dose and the possibility of side effects [9].

Chitosan has emerged as a prominent nanomaterial for biomedical applications due to its biocompatibility, biodegradability, abundant availability, and low cost [10-12]. Native chitosan has poor water solubility at physiological pH and is limited in its biomedical application [13, 14]. This can be improved by chemical modification, such as by introducing a hydrophobic or a hydrophilic moiety [15], an alkyl group [16], cholesterol [17], or poly(ethylene glycol) (PEG) [18-24].

### II. Conventional Chitosan Drug Delivery Systems

Currently, increased bioavailability of drugs is usually achieved by biologically active substance modification resulting from a chemical bond formation with poly(ethylene glycol) or PEGylation. This protocol requires chemical intervention to attach the PEG, which often involves multiple synthetic procedures. On the contrary, the supramolecular approach, subject to this invention does not require chemical bond formation for the drug coating and shows the best performance among the materials of this kind.

Chitosan has been reported to be part of a hydrophilic micelle shell building blocks comprising a water-soluble fragment that may be covalently bound to a hydrophobic polymer block comprising, for example, poly(e-caprolactone). Even if drugs are covalently attached to a hydrophobic polymer block, the chitosan has been reported as only part of a solubilizing shell. Although it is not necessary to understand the mechanism of an invention, it is believed that the presently disclosed linear-dendritic complexes places chitosan in the hydrophobic interior, as opposed to the exterior shell, where it serves as a carrier platform for drugs and other substances. In most cases, core-chitosan shell micelles are produced after a hydrophobic molecule was grafted onto a chitosan polymer. These compositions are unlike the supramolecular complexes discloses herein, which are much more uniform throughout and contain an even distribution of chitosan due to hydrophobic interactions between a cholanic moiety and dendrons of the branched polymers.

It has also been reported that chitosan may undergo an ionic interaction with oleic acid and/or linoleic acid or may be dispersed with PLA micelles. A hydrophobic modification of glycol chitosan has been published. While the synthesis of the linear-dendritic (LD) copolymers has been published, a combination of chitosan and LD copolymers into a single hydrophobic core has not been reported.

### III. Supramolecular Complexes Comprising a Chitosan Core

In distinction to the known micellar drug delivery systems, which are based on Pluronics® triblock copolymers, poly(ester)-block-poly(ether)s or poly(ethylene oxide)-block-poly(peptide)s, some embodiments of the presently disclosed chitosan supramolecular complex system has superior encapsulation efficiency and binding capacity. Additional advantages include, but are not limited to, the capability to encapsulate simultaneously a ‘cocktail’ of substances which might include, but are not limited to, biological fluorescent markers, mutually complementary drug molecules, oligopeptides and/or genetic sequences. Another advantage of this supramolecular system is the capability to modulate the surface moieties for targeted drug delivery and cell labeling.

In one embodiment, the present invention contemplates a method for delivering at least one compound to specific disease targets using a supramolecular chitosan complex. In one embodiment, the disease targets include, but are not limited to, metastatic cancers such as osteosarcoma and breast cancer. In one embodiment, the supramolecular chitosan complex comprises a targeting agent. In one embodiment, the targeting agent includes, but is not limited to, an anti-mmp14 antibody and/or folic acid. In one embodiment, the compound comprises at least one drug. In one embodiment, the at least one drug includes, but is not limited to, doxorubicin, ansamitocin-P3 and/or paclitaxel. In one embodiment the compound comprises at least one cellular marker. In one embodiment the at least one cellular marker is a fluorescent probe. In one embodiment, the fluorescent probe includes, but is not limited to, Cy5.5, Cy3 and/or fluorescein. In one embodiment, the compounds are delivered to a human osteosarcoma cell. In one embodiment, the delivering comprises an intracellular delivery.

In some embodiments, the supramolecular chitosan complex contemplates by this invention may comprise of one, two or more types of linear-dendritic block copolymers containing poly(ethylene glycol) or poly(ethylene oxide) of molecular weight 3,000 to 15,000 Da and dendritic poly(3,5-dihydroxybenzyl alcohol) of molecular weight 728 and 1577 Da.

In some embodiments, linear-dendritic copolymers containing specific end-groups like folic acid, which can be recognized by specific cell receptors like those in breast cancer tumor cells, can be added without disturbing the integrity of the micelles or their loading capacity.

In some embodiments, a supramolecular chitosan complex as contemplated herein may further comprise one or more hydrophobic substances including, but not limited to, polysaccharide-based micelles comprising a chemical conjugation of a hydrophobic moiety including, but not limited to, a 5-β-cholanic acid (e.g., approximately 360 Da), a hydrophilic glycol chitosan (e.g., approximately 250,000 Da), a fluorescent bio-marker (e.g., fluorescein), anticancer drugs (e.g., Doxorubicin or Paclitaxel), and/or an anti-inflammatory agent (e.g., curcumin), either singly or in combination.

In contrast to other drug delivery systems, the present invention contemplates that all “biologically active agents” (e.g., drugs and/or fluorescent markers) may be encapsulated within the chitosan hydrophobic core of the micelles without bond formation, for example, as free and unattached molecules. Consequently, release of these agents are triggered not by a bond cleavage, but by a change in the environment (e.g., for example, movement from inside the cell to outside the cell).

In distinction to other known micellar drug delivery systems, which are usually based on Pluronics® triblock copolymers, poly(ester)-block-poly(ether) or poly(ethylene oxide)-block-poly(peptide), embodiments of the present invention have superior encapsulation efficiency and binding capacity. Especially attractive is the possibility to encapsulate simultaneously a “cocktail” of substances, which might include, but are not limited to biological markers, mutually complementary drug molecules, oligopeptides and/or genetic sequences. Another useful characteristic of the supramolecular chitosan complex described herein is the possibility to modulate the surface functionalities in the corona for targeted drug delivery and cell labeling.

In some embodiments, the supramolecular chitosan complex core comprises cholanic acid, that may be either evenly or randomly dispersed within the core. Although it is not necessary to understand the mechanism of an invention, it is believed that 5-β-cholanic acid may impart hydrophobic properties to an otherwise hydrophilic molecule (e.g., for example, glycol chitosan). The resultant cholanic acid complex may result in a self-assembly of a micellar product when dispersed in water. See, Chin et al., “Evaluation of physicochemical characteristics of hydrophobically modified glycol chitosan nanoparticles and their biocompatibility in murine osteosarcoma and osteoblast-like cells” J Nanotech and Smart Mat 1:1-7 (2014). Hydrophobic molecules other than 5-beta-cholanic acid may also be used.

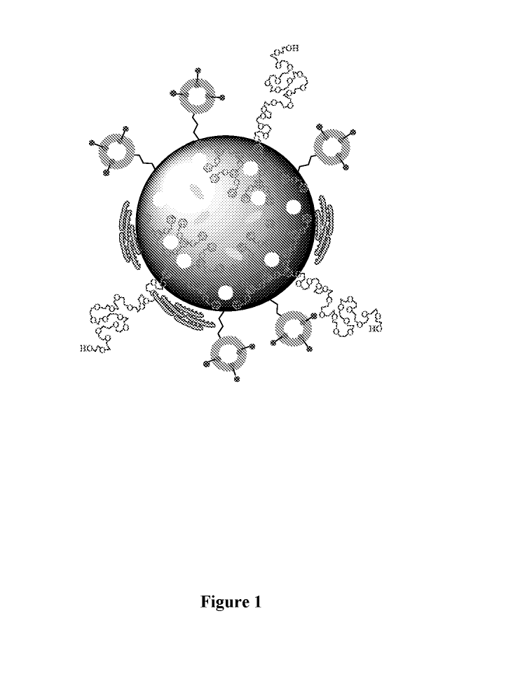

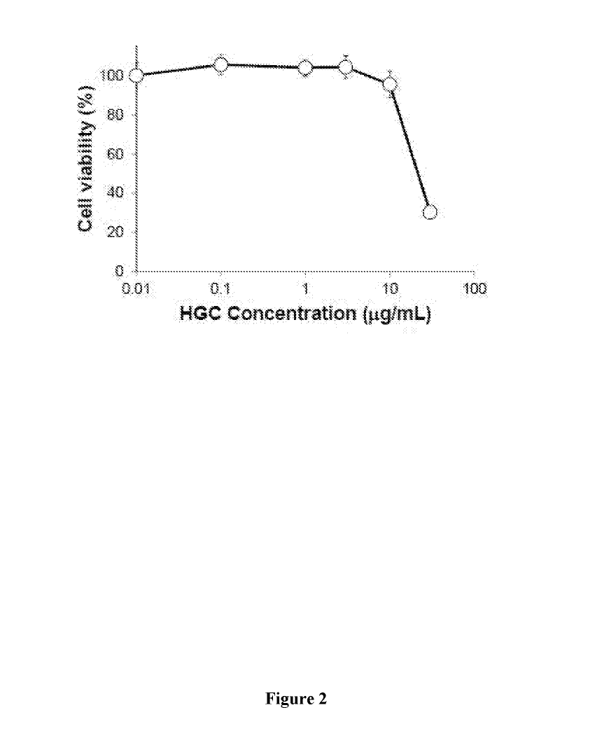

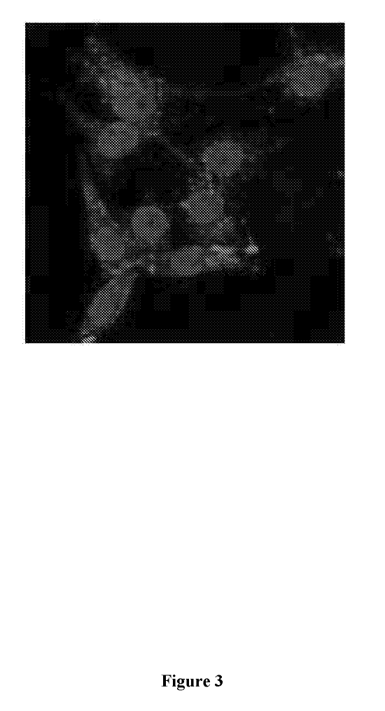

Biologically active substances can be incorporated (loaded) by contacting the substances to pre-formed micelles in an aqueous solution followed by sonication. See, FIG. 1. Alternatively, the substances may be encapsulated during micelle formation by mixing together the copolymers, chitosan and hydrophobic substances, followed by addition of an aqueous solution and sonication. A schematic representation of the supramolecular complex is shown in FIG. 1.

In some embodiments, the hydrophobic chitosan core comprises a plurality of dendritic fragments from the linear-dendritic copolymers (e.g., non-covalent PEGylation). Although it is not necessary to understand the mechanism of an invention, it is believed that the linear-dendritic copolymers form hydrophobic branched dendrons that anchor at the surface of the hydrophobic chitosan particle and immobilize the chitosan to the core of the micelle. In some embodiments, the linear-dendritic copolymers are perfectly branched dendrons. It is also believed that the linear-dendritic co-polymers may serve as non-entangled, non-bonded, nano-porous “reservoirs” for other hydrophobic substances, such as fluorescent markers (prevent their photobleaching), and biologically active molecules including, but not limited to proteins, peptides, drugs, enzymes and/or others. The poly(ethylene oxide) linear chains of these copolymers may form a corona of the micelle, thereby stabilizing the complex and making it water-soluble. One added benefit of the linear-dendritic chains is that they may be non-immunogenic, and consequently may not be detectable by cellular immune receptors thereby facilitating intracellular transport of the micelles into a cells cytoplasmic interior.

In some embodiments, the linear-dendritic polymer hydrophilic chains are not covalently bound to any substances, including but not limited to a drug, a biosensor, a cell marker or any other bioactive substance. In some embodiments, a cocktail comprising a plurality of different hydrophobic substances may be loaded into the hydrophobic core of the presently contemplated supramolecular chitosan micelles with a much higher loading capacity than other previously reported surfactants and other amphiphilic (or amphipathic) liner-linear copolymers.

Another added benefit is that targeting agents can be conjugated at ends of the hydrophilic shell poly(ethylene oxide) polymers, which could target specific organs, tissues cells and/or other macrocellular targets (e.g., for example, proteins and nucleic acid sequences).

Performance characteristics of some embodiments of the supramolecular chitosan complex include, but are not limited to:

1) Materials comprising a plurality of self-assembling polymers including, but not limited to, dendrimers, dendritic copolymers and/or polysaccharides.

2) Improved intracellular delivery to dendritic cells as a result of facilitated uptake capacity and/or activation of dendritic cells.

3) Improved immune activation ability including but not limited to, lymphocyte activation and/or anti-tumor activation. For example, it is expected to observe an IC₅₀ for a supramolecular chitosan/doxorubicin complex in the treatment of mouse B cell lymphoma 38C13 at approximately 0.0049 μg/mL.

4) Improved stability at 4° C., either as an aqueous suspension/solution or as a lyophilized powder.

5) Improved therapeutic efficacy as a result of high cell-targeting efficiency. 6) Formulations compatible with intravenous injection administration.

7) Improved amounts of containable drug wherein in some embodiments of a micelle-forming polysaccharide, the drug-polymer molar ratio for a hydrophobic drug such as doxorubicin is typically 30:1.

8) An improved human safety threshold wherein the IC₅₀ values of a micelle-forming polysaccharide in human osteosarcoma cells are estimated to be approximately 23 μg/mL. Cell viability of the supramolecular system in mouse T splenocytes at 2 mg/mL is 195%

9) In some embodiments, the supramolecular chitosan complex is a vaccine.

10) Improved uptake of a micelle-forming polysaccharide in cultured cancer cells (e.g., for example, within 15-30 minutes).

### III. Drug Delivery Platforms

The present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention may comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear.

In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

### IV. Pharmaceutical Formulations

The present invention further provides pharmaceutical compositions (e.g., comprising the supramolecular complexes as described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years. For example, in one embodiment, DOX-loaded HGC/LDC micelles may be administered at 10 mg DOX/kg per dose. Then at a 4% drug loading, each dose would equate to 250 mg micelles/kg, where 3 doses a day would deliver approximately 750 mg micelles/kg of body weight. These doses correspond with observed having anti-tumor activity in mice treated with docetaxel (DTX)-loaded HGC ranging from 10 mg to 30 mg DTX/kg. Hwang et al., “Tumor targetability and antitumor effect of docetaxel-loaded hydrophobically modified glycol chitosan nanoparticles” J Controlled Release 128:28-31 (2008).

### V. Antibodies

The present invention provides antibodies (i.e., for example, polyclonal or monoclonal) to be attached to a supramolecular chitosan complex. In one embodiment, the present invention provides monoclonal antibodies that specifically bind to any one of a plurality of disease biomarkers.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO2 gas. The antibody titer of the supernatant of a hybridoma culture can be measured according to the same manner as described above with respect to the antibody titer of the anti-protein in the antiserum. Separation and purification of a monoclonal antibody can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to a hapten in a weight ratio of about 0.1 part to about 20 parts, preferably, about 1 part to about 5 parts per 1 part of the hapten. In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times. The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a protein expressed resulting from a virus infection (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### EXPERIMENTAL

### Example I

### Construction Elements of Supramolecular Chitosan Complexes

1. Linear-dendritic block copolymers with linear block of methoxy-poly(ethylene glycol) having molecular weight of 1,800-2,800 Da and a second-generation dendritic block of poly(3,5-dihydroxybenzyl alcohol) having molecular weight of 728 Da.
2. Linear-dendritic block copolymers with linear block of methoxy-poly(ethylene glycol) having molecular weight of 4,800-5,800 Da and third generation dendritic block of poly(3,5-dihydroxybenzyl alcohol), having molecular weight of 1577 Da.
3. Linear-dendritic block copolymers with linear block of poly(ethylene oxide) having molecular weight of 1,800-2,800 Da and a second-generation dendritic block of poly(3,5-dihydroxybenzyl alcohol), having molecular weight of 728 Da.
4. Linear-dendritic block copolymers with linear block of poly(ethylene oxide) having molecular weight of 10,800-15,800 Da and a third-generation dendritic block of poly(3,5-dihydroxybenzyl alcohol), having molecular weight of 1577 Da.
5. Dendritic-linear-dendritic block copolymers with linear block of poly(ethylene glycol) having molecular weight of 4,000-6,000 Da and a second-generation dendritic block of poly(3,5-dihydroxybenzyl alcohol), having molecular weight of 728 Da.
6. Dendritic-linear-dendritic block copolymers with linear block of poly(ethylene glycol) having molecular weight of 8,500-11,500 Da and a third-generation dendritic block of poly(3,5-dihydroxybenzyl alcohol), having molecular weight of 1577 Da.
7. Dendritic-linear-dendritic copolymers with linear block of poly(ethylene oxide) having molecular weight of 10,800-15,800 Da, one third-generation dendritic block of poly(3,5-dihydroxybenzyl alcohol) having molecular weight of 1577 Da and one second- or third generation block of poly(3,5-dihydroxybenzyl alcohol) having molecular weights of 728 or 1577 Da, respectively.

### Example II

### Encapsulation of Hydrophobic Substances in Chitosan Supramolecular Complexes

1. Initial formation of the supramolecular system in water or any other non-toxic polar solvent using quantities of linear-dendritic or dendritic-linear-dendritic copolymers ranging from 1.2 mg/mL to 6.7 mg/mL, followed by addition of the hydrophobic substance (modified chitosan and/or fluorescent marker and/or drug) in solid (powder) form and sonication from 0.5 to 6 h.
2. Initial dry mixing of the linear-dendritic or dendritic-linear-dendritic copolymers and the hydrophobic substance (modified chitosan and/or fluorescent marker and/or drug), followed by addition of water or any other non-toxic polar solvent under sonication.
3. Initial solution of the linear-dendritic or dendritic-linear-dendritic copolymers and the hydrophobic substance in good non-toxic solvent, which is miscible with water and gradual addition of the resulting homogeneous solution to large (20-50×) excess of water.

### Example III

### Chitosan Modification

The hydrophobic modification of glycol chitosan was achieved via the formation of an amide bond between glycol chitosan and 5-beta-cholanic acid. Specifically, the carboxylic groups on 5-beta-cholanic acid were first activated by N-hydroxy-succinimide (NETS) and N-(3-dimethylaminopropyl)-N′-ethylcarbodiimide hydrochloride (ECD) before adding glycol chitosan (250,000 Da). The mixture was then stirred, dialyzed, centrifuged and lyophilized. To fluorescently label the hydrophobically modified glycol chitosan, Cy5.5-NHS or Cy3-NHS was first dissolved in dimethyl sulfoxide (DMSO) and added dropwise to a solution of 5-beta-cholanic acid-conjugated glycol chitosan (also in DMSO). The resulting vividly blue solution was then stirred, dialyzed and lyophilized.

The particle size distribution of the chitosan suspension was determined at 25° C. by Dynamic Light Scattering (DLS, Zetasizer Nano, Malvern Instruments Ltd., Westborough, Mass.). To evaluate the biocompatibility of chitosan nanomicelles, we conducted a viability assay using a human osteosarcoma cancer cell line. Specifically, MG63 cells were seeded at a density of 7,500 cells per cm² and allowed to attach for 24 hours in a humidified incubator (37° C., 5% CO2). Nanomicelle suspensions were prepared in serum free culture medium, followed by treatment with sonication to homogenize the suspension. Once the suspensions appeared homogenous, they were passed through a 0.8 μm and a 0.2 μm syringe filter to remove large aggregates and biological contaminants. The nanomicelles were added to the supernatant of each cancer cell culture well, and the cells were returned to the incubator and maintained for up to 72 hours. At each time interval, their viability was assessed using the MTS (3-(4,5-dimethylthiazol-2-yl)-5-(3-carbox-ymethoxyphenyl)-2-(4-sulfophenyl)-2H-tetrazolium) colorimetric assay.

### Example IV

### Intracellular Localization of Chitosan Nanomicelles

To investigate the intracellular fate of the chitosan nanomicelles, MG63 cells were plated on 12 mm (diam.) glass coverslips at a starting concentration of 70,000 cells/cm2 and let attached overnight. The media was replaced with media containing Cy5.5-labeled chitosan nanomicelles. At the desired time points, cells were washed twice with phosphate buffered saline (PBS), fixed in 3.7% formaldehyde and mounted on microscope glass slides with Fluoromount G. The samples were observed under a Leica TCS SP5 Confocal Laser Scanning Microscope.

### Example V

### Synthesis of 5β-Cholanic Acid-Conjugated Chitosan Nanoparticles

Water-soluble chitosan (500 mg) is first dissolved in deionized (DI) water (60 mL). The carboxylic acids groups of 5β-cholanic acid are then activated with 1.5 equivalents of N-hydroxysuccinimide (NHS) and N-(3-Dimethylaminopropyl)-N′-ethylcarbodiimide hydrochloride (EDC) dissolved in methanol (60 mL). The hydrophobic modification of chitosan is carried out by slowly combining to two solutions under stirring for 24 hours at room temperature to permit the 5β-cholanic acid to conjugate to the chitosan through the formation of amide linkages. The solution is dialyzed in a dialysis membrane (10 kDa molecular weight cut off) for 24 hours against a water/methanol mixture (1:4 v/v) and the following 24 hours against DI water. The purified solution is lyophilized and ground into a fine powder. The conjugates are suspended in either DI water or phosphate-buffered saline (PBS) at a concentration of 1 mg/mL and probe-type sonicated (S-450D Sonifier, Branson Ultrasonics, Danbury, Conn.) at 90 W for two minutes. This sonication step is repeated three times to ensure that the self-assembly of the conjugates into nanoparticles.

### Example VI

### Cy5.5—Labeling of Chitosan Nanoparticles

Cy5.5 is one of the most prominent near infrared dyes which can be used for noninvasive imaging of live animals, but to some extent its usage is restricted by insufficient hydrophilicity [25]. To fluorescently label chitosan, Cy5.5-NHS (1 mg) is first dissolved in dimethyl sulfoxide (DMSO, 250 μL) and subsequently added dropwise to a bulk solution of 5β-cholanic acid-conjugated chitosan. The resulting vividly blue solution is then stirred for 6 hours at room temperature, shielded from light. The Cy5.5-labeled conjugate solution is then dialyzed (10 kDa molecular weight cut off) in DI water to remove unreacted Cy5.5 molecules. Following 2 days of dialysis, the solution is lyophilized and ground to produce a blue powder. In order to protect the fluorescent properties of the Cy5.5 dye, these processes are performed in the dark. The Cy5.5-labeled conjugates are then probe-type sonicated as described above to form self-assembled fluorescent nanoparticles. Prior to cell culture, the nanoparticle solutions are passed through a 0.2 μm syringe filter to remove aggregates and biological contaminants.

### Example VII

### Characterization of Chitosan Nanoparticles

The size and zeta potential of the chitosan nanoparticles (CNP) are measured at 25° C. using dynamic light scattering (DLS) with a 633 nm laser (Malvern Zetasizer Nano-ZS; Malvern, Worcestershire, UK). The zeta potential is the measure of surface charge of nanoparticles and is a particularly influential parameter to understand the electrostatic interaction between nanoparticles. This characteristic is also suggestive of a nanoparticle's affinity to penetrate negatively charged biological membranes. CNP suspensions prepared in water at a concentration of 1 mg/mL are further sonicated in a water bath for 10 minutes at room temperature prior to DLS measurements. Additionally, a separate bath of CNP suspension are stored in water at 4° C. and particle size are measured using the DLS over the course of 10 days to determine the size stability. Average values are calculated from a minimum of three measurements. Standard error are calculated by dividing the standard deviation by the square root of n, the sample size.

### Example VIII

### Cell Viability

After 72 hr of incubation, the cells are fixed with 3.7% paraformaldehyde and rinsed two times with PBS before being fixing with 3.7% formaldehyde. Cells are rinsed two additional times with PBS and stained with the nuclear dye, 4′,6-diamidino-2-phenylindole (DAPI). The area density of the cells is determined by enumerating DAPI stained nuclei on an inverted fluorescence microscope (Olympus IX51).

### Example IX

### Supramolecular Complex Cell Labeling

This example provides data showing astrocyte 1321N1 cell culture labeling using a supramolecular complex encapsulating a acrylodan-labeled C-terminal phospholipase D (PLD) peptide.

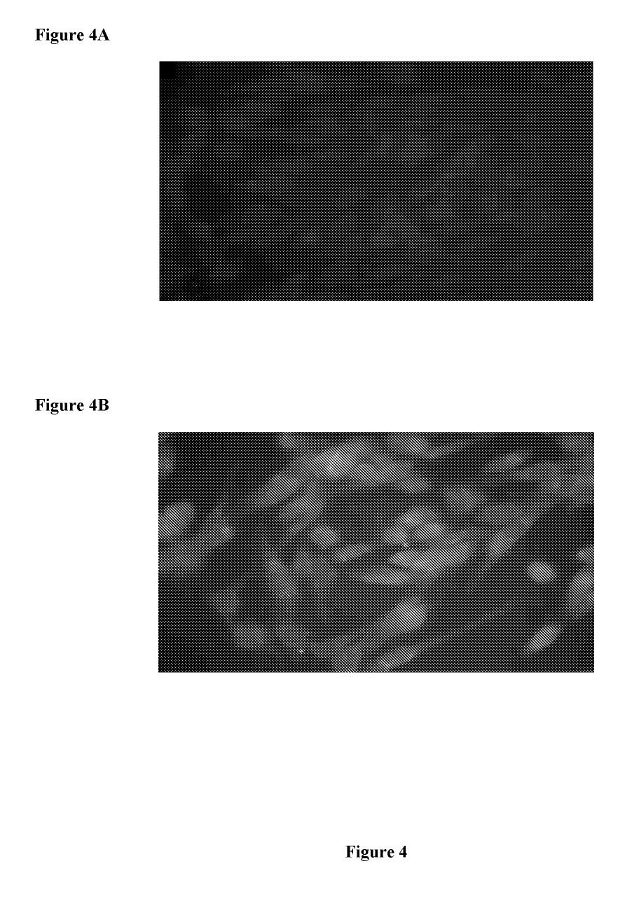

Control Astrocyte Image—A supramolecular complex without any encapsulated peptide was successfully delivered into the intracellular space of astrocyte cells while in the presence of glycerol. The image was exposed twice to achieve even faint picture of cells. The glycerol added causes faint auto-fluorescence seen in these control cells. See, FIG. 4A.

Labeled Astrocyte Image—A supramolecular complex encapsulating acrylodan-labeled C-terminal PLD peptide was successfully delivered into the intracellular space of astrocyte cells while in the presence of glycerol. The image demonstrates a highly distinct image of the astrocyte cell culture. See, FIG. 4B.

Both Control and Labeled Astrocyte images were taken at 400× magnification using conventional light microscopy with a DAPI filter set.

### Example X

### Cytotoxicity Assay

The assay was performed using [³H]-thymidine incorporation. Cells (1×10⁴-5×10⁵) in RPMI 1640 medium were seeded into 96-well FB-tissue culture plates (NUNC, Denmark). The specific cell types used in this assay were as follows:

Cancer Cell Lines
Mouse origin: EL 4 T cell lymphoma, American Type Culture Collection
Human origin: SW620 colorectal carcinoma (metastatic), American Type Culture Collection
Both cell lines were maintained in RPMI 1640 culture medium

Mouse T Splenocytes
The cells were isolated from 3-month-old females of inbred strain Balb/c.

Cancer cell lines were cultured without any additional stimulation; T splenocytes were stimulated with T cell mitogen-concavalin A (Con A; 1.25 μg/well, Pharmacia, Sweden). Different concentrations of samples were added to the wells to reach a final volume of 250 μL. The plates were incubated with the samples for 24 to 72 h at 37° C. in a humidified atmosphere (5% CO₂). Triplicate cells were used for each test condition.

The collected data can be summarized as follows:
1. SW620 Colorectal carcinoma cells (metastatic)
   [G2-PEG5k-G2], IC₅₀>2 mg/mL; Cell Viability at 2 mg/mL=60%
   [G3-PEG11k-G3], IC₅₀>2 mg/mL; Cell Viability at 2 mg/mL=100%
2. EL4 Lymphoma T cells
   [G2PEG5kG2], IC₅₀>2 mg/mL; Cell Viability at 2 mg/mL=88%
   [G3-PEG11k-G3], IC₅₀>2 mg/mL; Cell Viability at 2 mg/mL=74%
3. Mouse T splenocytes
   [G2PEG5kG2], IC₅₀=0.59 mg/mL; Cell Viability at 2 mg/mL=19%
   [G3PEG11kG3], IC₅₀>2 mg/mL; Cell Viability at 2 mg/mL=195%
4. SW620 Colorectal carcinoma cells (metastatic)
   [G3-PEG11k-G3/Doxorubicin], IC₅₀=0.015 μg/mL;
5. EL4 Lymphoma T cells
   [G3-PEG11k-G3/Doxorubicin], IC₅₀=0.006 μg/mL
      This cytostatic activity is 185 times more effective than the one reported with other polymeric micelles for the same EL4 lymphoma T cell line (ref. 26).

### Example XI

### Improved Anti-Cancer Effect of HGC-LDC Complex

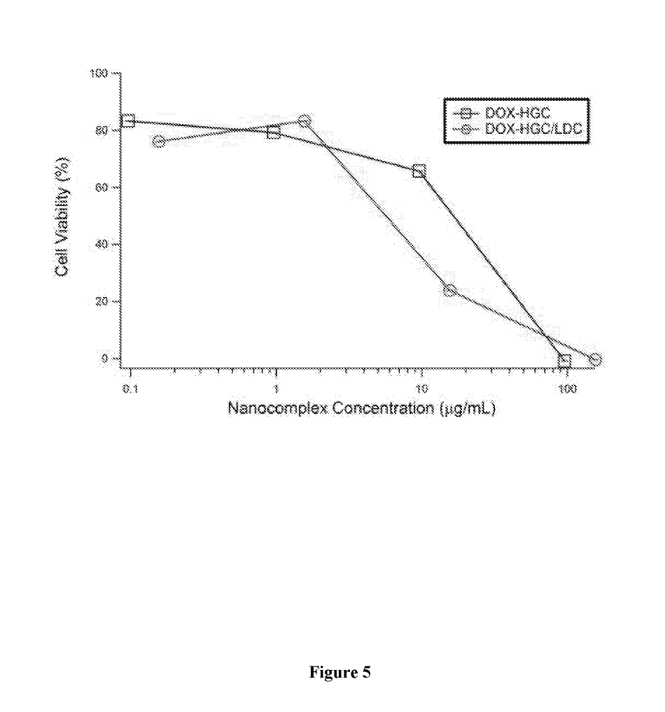

This example provides data showing that a supramolecular complex of hydrophobically modified glycol chitosan with linear-dendritic copolymer, or HGC-LDC, enhanced the cytotoxic effect of the anticancer drug doxorubicin (DOX), when compared to DOX that was encapsulated in HGC alone. To evaluate the cytotoxicity of DOX-polymer nanocomplex, HGC and LDC were complexed at a mass ratio of 1:1 and DOX was loaded at 4% (w/w). The nanocomplexes were applied to a culture of 4T1 murine carcinoma cells for 3 days, and their viability was assessed using a colorimetric assay (CellTiter 96® AQueous One Solution Cell Proliferation Assay, Promega). As shown in FIG. 5, DOX in HGC-LDC inhibited breast cancer cell proliferation much more efficiently than DOX in HGC. The IC₅₀ values were calculated to be 8.4 μg/mL for DOX-HGC/LDC and 19.8 μg/mL for DOX-HGC, respectively. See, FIG. 5.

### REFERENCES

1) K. Cho, X. Wang, S. Nie, Z. Chen, D. M. Shin (2008) Therapeutic nanoparticles for drug delivery in cancer, Clinical Cancer Research. 14: 1310-1316.
2) K. Greish (2007) Enhanced permeability and retention of macromolecular drugs in solid tumors: A royal gate for targeted anticancer nanomedicines, J Drug Target. 15: 457-464.
3) S. Pasha, K. Gupta, (2010) Various drug delivery approaches to the central nervous system, Expert Opin Drug Deliv. 7: 113-135.
4) K. Kim, J. H. Kim, H. Park, Y.-S. Kim, K. Park, et al. (2010) Tumor-homing multifunctional nanoparticles for cancer theragnosis: Simultaneous diagnosis, drug delivery, and therapeutic monitoring, J Control Release. 146: 219-227.
5) M. Morille, C. Passirani, A. Vonarbourg, A. Clavreul, J. P. Benoit (2008) Progress in developing cationic vectors for non-viral systemic gene therapy against cancer, Biomaterials. 29: 3477-3496.
6) Y. Matsumura, H. Maeda (1986) A new concept for macromolecular therapeutics in cancer-chemotherapy—mechanism of tumoritropic accumulation of proteins and the antitumor agent smancs, Cancer Res. 46: 6387-6392.
7) Y. Kim, M. H. Pourgholami, D. L. Morris, M. H. Stenzel (2011) Triggering the fast release of drugs from crosslinked micelles in an acidic environment, Journal of Materials Chemistry, 21: 12777-12783.
8) P. D. Thornton, R. J. Mart, R. V. Ulijn (2007) Enzyme-responsive polymer hydrogel particles for controlled release, Advanced Materials, 19: 1252-1256.
9) M. Oishi, Y. Nagasaki (2010) Stimuli-responsive smart nanogels for cancer diagnostics and therapy, Nanomedicine. 5: 451-468.
10) Q. Gan, T. Wang, C. Cochrane, P. McCarron (2005) Modulation of surface charge, particle size and morphological properties of chitosan-TPP nanoparticles intended for gene delivery, Colloids Surf B Biointerfaces. 44: 65-73.
11) J. Kumirska, M. Czerwicka, Z. Kaczynski, A. Bychowska, K. Brzozowski, (2010) Application of Spectroscopic Methods for Structural Analysis of Chitin and Chitosan, Mar Drugs. 8: 1567-1636.
12) T. A. Sonia, C. P. Sharma (2011) Chitosan and Its Derivatives for Drug Delivery Perspective, Chitosan for Biomaterials I. 243: 23-53.
13) J. H. Park, Y. W. Cho, H. Chung, I. C. Kwon, S. Y. Jeong (2003) Synthesis and characterization of sugar-bearing chitosan derivatives: Aqueous solubility and biodegradability, Biomacromolecules, 4: 1087-1091.
16) Sashiwa H, Kawasaki N, Nakayama A, Muraki E, Yamamoto N (2002) Chemical modification of chitosan. 13. Synthesis of organosoluble, palladium adsorbable, and biodegradable chitosan derivatives toward the chemical plating on plastics, Biomacromolecules. 3: 1120-1125.
17) Y. S. Wang, L. R. Liu, Q. Jiang, Q. Q. Zhang (2007) Self-aggregated nanoparticles of cholesterol-modified chitosan conjugate as a novel carrier of epirubicin,
European Polymer Journal, 43: 43-51.
18) N. Bhattarai, J. Gunn, M. Zhang (2010) Chitosan-based hydrogels for controlled, localized drug delivery, Adv Drug Deliv Rev. 62: 83-99. 19) Y. I. Jeong, D. G. Kim, M. K. Jang, J. W. Nah (2008) Preparation and spectroscopic characterization of methoxy poly(ethylene glycol)-grafted water-soluble chitosan, Carbohydr Res. 343: 282-289.
20) T. Ouchi, H. Nishizawa, Y. Ohya (1998) Aggregation phenomenon of PEGgrafted chitosan in aqueous solution, Polymer, 39: 5171-5175.
21) Dong-Gon Kim, Young-Il Jeong, Jae-Woon Nah (2007) All-trans retinoic acid release from polyion-complex micelles of methoxy poly(ethylene glycol) grafted chitosan. Journal of Applied Polymer Science. 105: 3246-3254.
22) S. R. Mao, X. T. Shuai, F. Unger, M. Wittmar, X. L. Xie, et al. (2005) Synthesis, characterization and cytotoxicity of poly(ethylene glycol)-graft-trimethyl chitosan block copolymers, Biomaterials. 26: 6343-6356.
23) Y. Q. Hu, H. L. Jiang, C. N. Xu, Y. J. Wang, et al. (2005) Preparation and characterization of poly(ethylene glycol)-g-chitosan with water- and organosolubility, Carbohydrate Polymers. 61: 472-479.
24) H. Saito, X. D. Wu, J. M. Harris, A. S. Hoffman (1997) Graft copolymers of poly(ethylene glycol) (PEG) and chitosan, Macromolecular Rapid Communications. 18: 547-550.
25) S. Luo, E. Zhang, Y. Su, T. Cheng, C. Shi (2011) A review of NIR dyes in cancer targeting and imaging, Biomaterials. 32: 7127-7138.
26) D. Vetvicka, M. Hruby, O. Hovorka, T. Etrych, Vetrik, L. Kovar, M. Kovar, K. Ulbrich, B. Rihova (2009) Biological Evaluation of Polymeric Micelles with Covalently Bound Doxorubicin, Bioconjugate Chemistry. 20: 2090-2097

We claim:

1. A supramolecular complex comprising a hydrophobic core encapsulated by a hydrophilic polymer shell, wherein said hydrophobic core comprises at least one chitosan nanoparticle anchored by at least one linear-dendritic co-polymer.

2. The supramolecular complex of claim 1, wherein said chitosan is a covalently modified chitosan.

3. The supramolecular complex of claim 1, wherein said hydrophobic core further comprises an unattached compound.

4. The supramolecular complex of claim 3, wherein said unattached compound is selected from the group consisting of a drug, a cell marker and a biosensor.

5. The supramolecular complex of claim 1, wherein said hydrophilic polymer shell comprises a targeting agent.

6. The supramolecular complex of claim 5, wherein said targeting agent comprises avidin and a streptavidin-conjugated antibody.

7. The supramolecular complex of claim 6, wherein said streptavidin-conjugated antibody has specific affinity for a disease biomarker.

8. The supramolecular complex of claim 7, wherein said disease biomarker is selected from the group consisting of a protein, a peptide, a polypeptide and nucleic acid sequence.

9. A method, comprising:
a. providing:
i) a supramolecular complex comprising a hydrophobic core encapsulated by a hydrophilic polymer shell, wherein said hydrophobic core comprises at least one chitosan nanoparticle anchored by at least one linear-dendritic co-polymer and a compound;
ii) a patient exhibiting at least one symptom of a medical condition;
b. administering said supramolecular complex to said patient under conditions such that said at least one symptom is reduced.

10. The method of claim 9, wherein said medical condition comprises a metastatic cancer.

11. The method of claim 10, wherein said metastatic cancer is selected from the group consisting of osteosarcoma and breast cancer.

12. The method of claim 9, wherein said hydrophilic shell further comprises a targeting agent specific for a biomarker of said medical condition.

13. The method of claim 12, wherein said targeting agent is selected from the group consisting of an antibody having specific affinity for said biomarker

14. The method of claim 10, wherein said compound is selected from the group consisting of a drug, a cellular marker and a biosensor.

15. The method of claim 11, wherein said drug is selected from the group consisting of doxorubicin, ansamitocin-P3 and paclitaxel.

16. The method of claim 9, wherein said administering comprises an intracellular delivery.
